(19) United States
(12) Patent Application Publication
(10) Pub. No.: US 2026/0212955 A1
(43) Pub. Date: Jul. 23, 2026

(54) MACHINE LEARNING BASED ENZYME EVOLUTION

(71) Applicant: Agency for Science, Technology and Research, Singapore (SG)

(72) Inventors: Sebastian MAURER-STROH, Singapore (SG); Jhoann Margarette Tristeza MIYAJIMA, Singapore (SG); Hao FAN, Singapore (SG); Shreyas SUPEKAR, Singapore (SG); Adrian Matthew Weng Kin MAK, Singapore (SG); Marco KLAEHN, Singapore (SG); Ee Lui ANG, Singapore (SG); Wan Lin YEO, Singapore (SG); Yee Hwee LIM, Singapore (SG); Wei Peng Dillon TAY, Singapore (SG)

(21) Appl. No.: 19/141,699

(22) PCT Filed: Dec. 14, 2023
(86) PCT No.: PCT/SG2023/050830

§ 371 Date: Jun. 20, 2025

(30) Foreign Application Priority Data
Dec. 21, 2022 (SG) 10202260537Q

(51) Int. Cl. G16B 20/50; G16B 30/10; G16B 40/20

(52) U.S. Cl. CPC G16B 20/50; G16B 30/10; G16B 40/20

(57) ABSTRACT

Method and systems for enzyme evolution by performing sequence conservation analysis followed by attribute analysis using a plurality of attribute analysis models on a plurality of mutated candidate enzyme sequences and selecting one or more optimal enzyme sequences. The attribute analysis model comprises any one of aggregation potential prediction model or allosteric effect prediction model or thermostability prediction model or reaction kinetic prediction model or catalytic activity prediction model.

### TECHNICAL FIELD

This disclosure generally relates to methods and systems for enzyme evolution.

### BACKGROUND

Enzymes are used in the food, agriculture, cosmetic, and pharmaceutical industries to control and speed up reactions in order to quickly and accurately obtain a valuable final product. In nature, enzymes evolve over time to improve fitness of organisms. The natural evolution of enzymes is a slow process that may take thousands or millions of years. Enzyme are usually proteins and small variations in the molecular structure of enzymes may result in significant variation in the effectiveness of the enzyme. Since the molecular structure of enzymes is subject to a great degree of variability, the evolution of enzymes to identify more effective or optimum enzymes presents a significant computational and/or experimental challenge.

Enzyme optimization for any chemical process is a slow, laborious and expensive process. Despite the advantages of biocatalysis (high selectivity, sustainability, one-pot cascade reactions), its application in the industry has been limited to the narrow scope of identified enzymes and its growth restricted by slow enzyme development speed (typically months or years), lack of telescoped enzymatic reactions, and poor support for continuous operation.

It would be desirable to overcome or alleviate at least one of the above-described problems, or at least to provide a useful alternative.

This background description is provided for the purpose of generally presenting the context of the disclosure. Contents of this background section are neither expressly nor impliedly admitted as prior art against the present disclosure.

### SUMMARY

In one embodiment, the present disclosure provides a system of enzyme evolution, the system comprising:
a memory accessible to processor(s), the memory comprising program code executable by the processors to:
perform sequence conservation analysis on an enzyme sequence dataset of a class of enzymes to select a plurality of candidate sequences in the sequence dataset;
simulate mutation in the plurality of candidate sequences to obtain a plurality of mutated candidate enzyme sequences;
perform a first attribute analysis using a first attribute analysis model on the plurality of mutated candidate enzyme sequences to estimate a first attribute value of each of the plurality of mutated candidate enzyme sequence;
select one or more optimal enzyme sequences from the plurality of mutated candidate enzyme sequences based on the estimated first attribute values.

Some embodiments relate to a system for enzyme evolution, the system comprising:
a memory accessible to processor(s), the memory comprising program code executable by the processors to:
perform sequence conservation analysis on an enzyme sequence dataset of a class of enzymes to select a plurality of candidate sequences in the sequence dataset;
simulate mutation in the plurality of candidate sequences to obtain a plurality of mutated candidate enzyme sequences;
perform attribute analysis using a plurality of attribute analysis models on the plurality of mutated candidate enzyme sequences to estimate a plurality of attribute values of each of the plurality of mutated candidate enzyme sequence;
select one or more optimal enzyme sequences from the plurality of mutated candidate enzyme sequences based on the estimated plurality of attribute values;
wherein each of the plurality of attribute analysis model comprise any one of:
aggregation potential prediction model to predict an aggregation potential of mutated candidate enzyme sequences; or
allosteric effect prediction model to predict allosteric effects on fitness of mutated candidate enzyme sequences;
thermostability prediction model to predict thermostability of mutated candidate enzyme sequences; or
reaction kinetic prediction model to predict kinetics of a reaction of the mutated candidate enzyme sequences; or
catalytic activity prediction model to predict catalytic activity of the mutated candidate enzyme sequences.

Some embodiments relate to a method for enzyme evolution, the method comprising:
performing sequence conservation analysis on an enzyme sequence dataset of a class of enzymes to select a plurality of candidate enzyme sequences in the sequence dataset, wherein the candidate enzyme sequences are viable candidates for subsequent evolution;
simulating mutation in the plurality of candidate sequences to obtain a plurality of mutated candidate enzyme sequences;
performing a first attribute analysis using a first attribute analysis model on the plurality of mutated candidate enzyme sequences to estimate a first attribute value of each of the plurality of mutated candidate enzyme sequence;
selecting one or more optimal enzyme sequences from the plurality of mutated candidate enzyme sequences based on the estimated first attribute values.

Some embodiments relate to a method for enzyme evolution, the method comprising:
performing sequence conservation analysis on an enzyme sequence dataset of a class of enzymes to select a plurality of candidate enzyme sequences in the sequence dataset, wherein the candidate enzyme sequences are viable candidates for subsequent evolution;
simulating mutation in the plurality of candidate enzyme sequences to obtain a plurality of mutated candidate enzyme sequences;
performing attribute analysis using a plurality of attribute analysis models on the plurality of mutated candidate enzyme sequences to estimate a plurality of attribute values of each of the plurality of mutated candidate enzyme sequence;
selecting one or more optimal enzyme sequences from the plurality of mutated candidate enzyme sequences based on the estimated plurality of attribute values;
wherein the each of the plurality of attribute analysis model comprise any one of: aggregation potential prediction model to predict an aggregation potential of mutated candidate enzyme sequences; or
allosteric effect prediction model to predict allosteric effects on fitness of mutated candidate enzyme sequences;
thermostability prediction model to predict thermostability of mutated candidate enzyme sequences; or
reaction kinetic prediction model to predict kinetics of a reaction of the mutated candidate enzyme sequences; or
catalytic activity prediction model to predict catalytic activity of the mutated candidate enzyme sequences.

### BRIEF DESCRIPTION OF THE DRAWINGS

Some embodiments of methods for enzyme optimization, in accordance with present disclosure, will now be described, by way of non-limiting example only, with reference to the accompanying drawings in which:

FIG. 1 illustrates a framework for an integrated predictive enzyme development platform;

FIG. 2 illustrates a class of oxidation reactions catalyzed by galactose oxidase (GOase);

FIG. 3 illustrates high throughput colorimetric assay utilizing 2,2′-azino-bis(3-ethylbenzothiazoline-6-sulfonic acid) (ABTS) and horseradish peroxidase (HRP) to quantify H₂O₂ as an indirect measure of galactose oxidase (GOase) activity;

FIG. 4 illustrates a correlation graph between ketone yield, as quantified by high performance liquid chromatography (HPLC) against natural logarithm of GOase enzyme activity value, ln(activity);

FIG. 5 illustrates a flowchart for methods of enzyme evolution;

FIG. 6 illustrates a confusion matrix of predicted vs experimental results for GOase variants;

FIG. 7 illustrates a series of GOase active site models derived from X-ray crystal structures;

FIG. 8 illustrates a reaction scheme showing oxidation of alcohol substrate by GOase at the active site;

FIG. 9 illustrates the correlation between measured free energy barriers, (DG^{†}ₑₓₚ) and predicted free energy barriers (DG^{†}_{MD}) of GOase rate constants of different mutants;

FIG. 10 illustrates the active site of M3-5 mutant with the R-isomer (left side) and S-Isomer (right side) of the S128 substrate (shown in stick representation);

FIG. 11 illustrates a flowchart of a pipeline of modelling, docking, rescoring and featurization;

FIG. 12 illustrates a GOase catalytic mechanism;

FIG. 13 illustrates a catalytic activity prediction from the XGBoost model;

FIG. 14 illustrates a graph (on left) of 10-fold cross validation of training data set comprising 2654 substrate-mutant pairs with a Pearson correlation score of 0.89, and another graph (on right) of blind test prediction for 402 substrate-mutant pairs with a Pearson correlation score of 0.90;

FIG. 15 illustrates linear graphs of test data sets breaking down each mutant specific profile—GOh1036, GOh1052, GOh2008, GOh2027, GOh2039, GOh2045 and GOh2070;

FIG. 16 illustrates a comparison table of activity between original backbone GOh1001b vs the new mutants found in 1^{st} and 2^{nd} round of mutagenesis via CNN predictions;

FIG. 17 illustrates three ortho-substituted substrates S155, S172 and S173; and

FIG. 18 illustrates the development of enzyme with improved features using an integrated predictive enzyme model.

FIG. 19 illustrates the development of enzyme with improved features using an integrated predictive enzyme model.

### DETAILED DESCRIPTION

Embodiments relate to systems and methods for enzyme evolution. In particular, embodiments incorporate machine learning operations on data related to enzyme structure and function of enzymes to identify enzyme variants with greater efficacy or desirable characteristics. Enzyme evolution according to the disclosure comprises a pre-analysis to focus the search/evolution. The pre-analysis stage may include a sequence search and substrate binding site domain analysis to identify a reduced set of target/candidate enzymes and/or select a panel of substrates. The pre-analysis stage may also include enzyme-substrate modelling to prioritize active site residues and select more desirable enzyme variants. The pre-analysis stage may also include sequence conservation analysis for mutagenesis prioritization.

A second phase of the methods of the embodiments comprises application of a single computational model or integration of various computational models to predict the attributes of enzyme variants and select a subset of enzyme variants with desirable attributes. The computational models may predict catalytic activity, kinetics, allostery, thermostability or aggregation of the enzyme variants. The one or more than one computational models identify promising enzyme variants in silico. The selected enzyme variants may be subsequently evaluated using high throughput experiments. Computation using the computational models and the high throughput experiments may be performed iteratively to accelerate the development of synthetic metabolic pathways.

A “protein” or “polypeptide” comprises a polymeric sequence of amino acid residues. The terms “protein” and “polypeptide” are used interchangeably herein. The single and 3-letter code for amino acids as defined in the IUPAC-IUB Joint Commission on Biochemical Nomenclature (JCBN) is used throughout this disclosure. It is also understood that a polypeptide can be coded for by more than one nucleotide sequence due to the degeneracy of the genetic code. A single or 3-letter amino acid code preceded by or followed by a number (e.g., “Tyr272” or “272Y”) refers to the amino acid at the position indicated by that number within the amino acid sequence of a wild-type protein. Mutations can be named by the one letter code for the parent amino acid, followed by a number and then the one letter code for the variant amino acid. For example, mutating glycine (G) at position 87 to serine(S) can be represented as “G087S” or “G87S”. Multiple mutations can be indicated by inserting a “−,” “+,” or “,” between the mutations. For example, mutations at positions 87 and 90 can be represented as either “G087S-A090Y” or “G87S-A90Y” or “G87S+A90Y” or “G087S+A090Y”.

As used herein, an “enzyme” refers to a protein that catalyzes chemical reactions. A “substrate” is a molecule upon which an enzyme acts. The “active site” is the region of an enzyme where one or more substrate molecules bind and undergo a chemical reaction. The enzyme active site may comprise amino acid residues that bond temporarily with a substrate and/or amino acid residues that catalyze a reaction of that substrate. The arrangement of amino acids within the active site is generally unique to each enzyme, and this arrangement is partly responsible for determining the type of reaction an enzyme catalyzes, and also the range of substrates that the enzyme recognizes (i.e., the substrate specificity of the enzyme). Modification of amino acid residues in the active site may alter the substrate specificity of an enzyme and/or the kinetics of a reaction catalyzed by that enzyme.

The term “allosteric effect” as used herein refers to the impact on enzyme activity due to changes at a site on the enzyme that is topographically distinct from the enzyme's active site. The site at which a change may occur to induce an allosteric effect is termed an “allosteric site”. An allosteric site may be a native feature of an enzyme and may be where a natural effector molecule binds to regulate enzyme activity. An allosteric site may also be created by modifying amino acid residues outside of the enzyme active site. Such modifications may affect enzyme activity by, for instance, inducing conformational changes that in turn affect the conformation of the enzyme active site. An amino acid residue that has an allosteric effect is termed an “allosteric residue” herein.

Enzymes find various uses in the pharmaceutical, textile, cosmetics, food and biofuel industries. At various stages in any of these uses, enzymes may aggregate. Enzymes may also aggregate intracellularly when they are expressed recombinantly in a host cell. The term “aggregate” refers to a physical interaction between protein molecules that results in the formation of covalent or non-covalent dimers or oligomers, which may remain soluble, or form insoluble aggregates that precipitate out of solution. As used herein aggregates are abnormal protein associations that lack a natural function. In this respect they are distinguished from protein multimers and protein complexes that may assemble naturally and carry out a natural function. Protein aggregates herein may be unstructured (i.e., the aggregates are amorphous) or partially or fully structured. Amyloids are a class of protein aggregates characterized by a fibrillary morphology and extensive β-sheet secondary structural elements. The aggregation potential of a protein or enzyme may depend on extrinsic factors, such as the enzyme concentration in solution, solution pH, solution temperature, and whether the solution or protein is subject to mechanical stress. The aggregation potential of a protein or enzyme may also depend on intrinsic factors arising from its amino acid sequence, such as the presence of certain secondary structural elements, the presence of hydrophobic patches in its tertiary structure, or the presence of multiple disulfide bonds. Alterations to the amino acid sequence of an enzyme may thus modulate its aggregation potential. An enzyme that is less prone to aggregation may be structurally more stable and less prone to protein misfolding.

A protein with increased structural stability may also be more stable against denaturing agents or denaturing conditions, such as heat, detergents, chaotropic agents or extreme pH. The “thermostability” of a protein or enzyme, as used herein, should be taken broadly to refer to a protein's resistance to irreversible denaturation induced by thermal and/or chemical approaches including, but not limited to, heating, cooling, freezing, chemical denaturants, pH, detergents, salts, additives, proteases or temperature. Irreversible denaturation leads to the irreversible unfolding of the functional conformation of the enzyme, loss of enzyme activity and aggregation of the denatured enzyme. The terms “(thermo) stabilize”, “(thermo) stabilizing”, and “increasing the (thermo) stability of”, as used herein, apply to enzymes in a native environment (e.g., inside a cell) and to enzymes that are applied for industrial use.

As used herein a protein “structural model” is a representation of a protein's three-dimensional secondary, tertiary, and/or quaternary structure. A structural model may encompass, among others, X-ray crystal structures, NMR structures, theoretical protein structures, structures created from homology modeling, protein tomography models, and atomistic models built from electron microscopic studies. Typically, a “structural model” will not merely encompass the primary amino acid sequence of a protein, but will provide coordinates for the atoms in a protein in three-dimensional space, thus showing the protein folds and amino acid residue positions. The structural model analyzed may be an X-ray crystal structure, e.g., a structure obtained from the Protein Data Bank (PDB, https://www.rcsb.org/) or a homology model built upon a known structure of a similar protein. The structural model may be pre-processed before applying the methods of the present invention. For example, the structural model may be put through a molecular dynamics simulation to allow the protein side chains to reach a more natural conformation, or the structural model may be allowed to interact with solvent, e.g., water, in a molecular dynamics simulation. The pre-processing is not limited to molecular dynamics simulation and may be accomplished using any art-recognized means to determine movement of a protein in solution. An example of an alternative simulation technique is Monte Carlo simulation. Monte Carlo simulation may be performed using simulation packages or any other acceptable computing means. In certain embodiments, simulations to search, probe or sample protein conformational space may be performed on a structural model to determine movement of the protein.

A “theoretical protein structure” is a three-dimensional protein structural model which is created using computational methods often without any direct experimental measurements of the protein's native structure. A “theoretical protein structure” encompasses structural models created by ab-initio methods and homology modeling. A “homology model” is a three-dimensional protein structural model which is created by homology modeling, which typically involves comparing a protein's primary sequence to the known three-dimensional structure of a similar protein.

“Docking” as used herein, refers to the computational process for simulating and/or characterizing the binding of a computational representation of a low molecular-weight molecule (e.g., a substrate or ligand, low molecular weight is arbitrarily defined here as a molecular weight below 1000 daltons) to a computational representation of an active site of an enzyme. Docking is typically implemented in a computer system using a “docker” computer program. Typically, the result of a docking process is a computational representation of the molecule “docked” in the active site in a specific “pose”. A plurality of docking processes may be carried out between the same computational representation of a molecule and the same computational representation of an active site resulting in a plurality of different “poses” of the molecule in the active site. The evaluation of the structure, conformation, and energetics of the plurality of different “poses” in the computational representation of the active site can identify certain “poses” as more energetically favorable for binding between the ligand and the biomolecule.

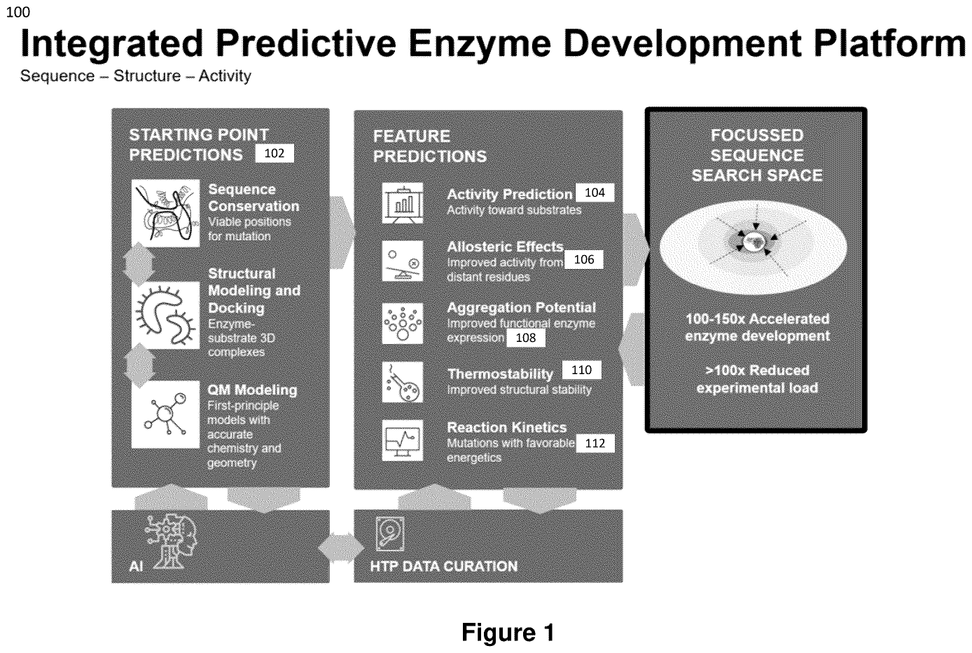

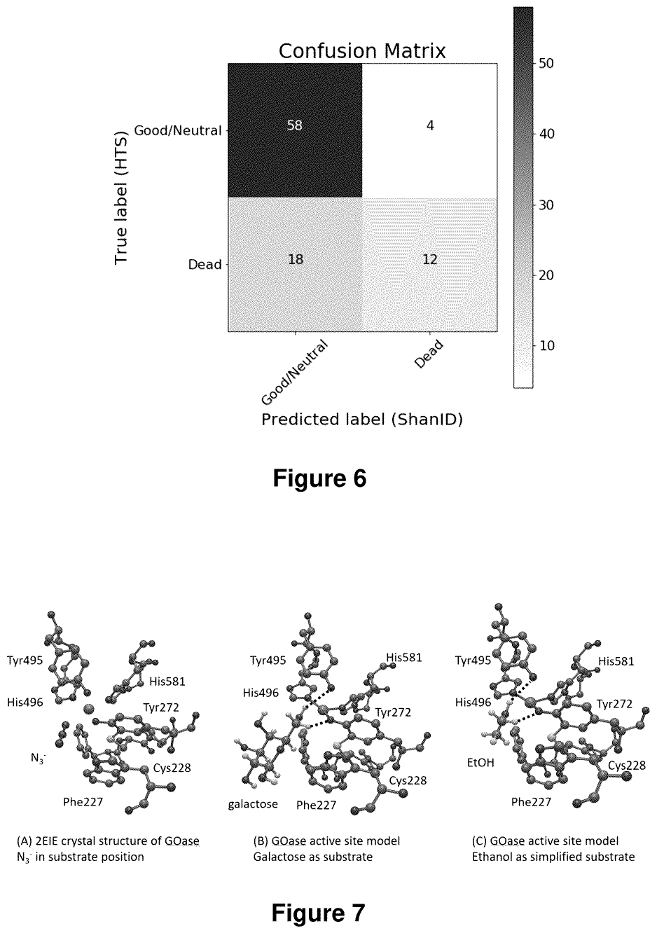

FIG. 1 discloses a framework of an integrative, predictive computational-experimental method to accelerate the enzyme optimization and development process 100. The method comprises: a high-throughput method to acquire large, curated datasets for developing the computational model (>4000 data points); a sequence conservation analysis model 102 to prioritize likely beneficial amino acid positions for investigation and reduce enzyme search space (also shown in FIG. 6); and a predictive model for optimizing enzyme characteristics such as activity 104, allosteric effects 106, aggregation potential 108, thermostability 110, and reaction kinetics 112 on enzyme fitness.

In some embodiments the method is able to, inter alia, predict the activity of enzyme variants for ortho-substituted substrates with Pearson correlation of about 0.74 against HPLC yield; shortlist enzyme variants with high thermostability with accuracy of about 75% (exemplary data in Table 3); and predict the aggregation potential of an enzyme variant with up to 62% accuracy (exemplary data in Table 1). The methods streamline and accelerate the enzyme development process by reducing extensive experimentation and time required to develop an optimal enzyme.

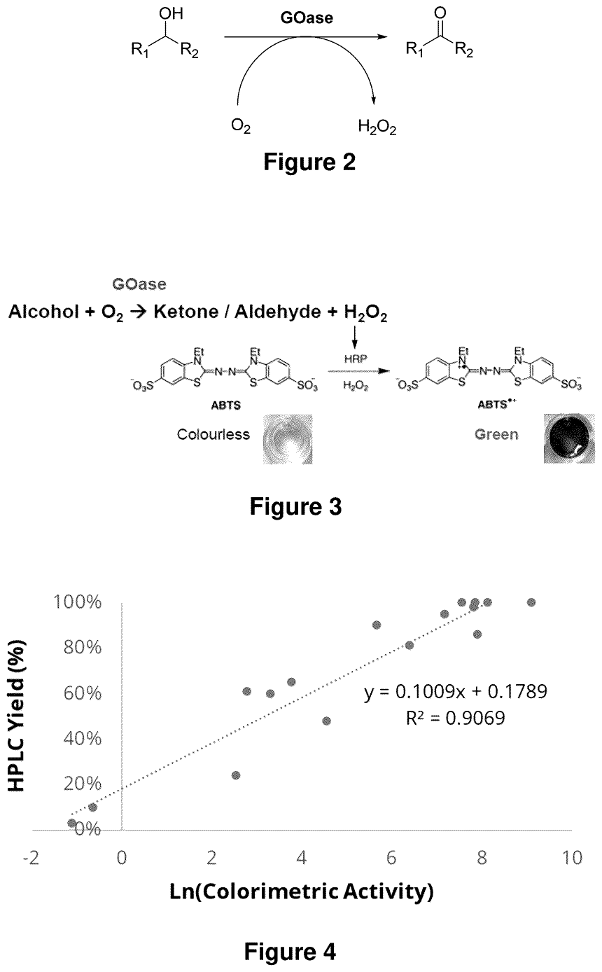

The methods have been used to engineer variants of the enzyme galactose oxidase (GOase), which catalyze the industrially important oxidation of primary and secondary alcohols to aldehydes and ketones respectively. FIG. 2 illustrates an example of one such reaction. While several aspects of the disclosed methods and systems have been described with reference to the GOase enzyme, the disclosure is not limited to evolution of GOase enzyme variants. The methods and systems of the disclosure may be applied to other enzyme classes to evolve or engineer other variants of other enzymes.

Galactose oxidase is a copper radical enzyme. Its catalytic site contains an oxidized copper center (Cu^{II}) coordinated by a tyrosyl radical that is post-translationally covalently cross-linked to a cysteine (CYS-TYR). The tyrosyl radical (TYR-O·) is non-trivial to set up for classical protein structural modeling and substrate docking. Furthermore, the protein-ligand scoring/rescoring functions used in computation mainly estimate substrate binding affinities but are not directly related to the experimental readouts such as product yields. Moreover, the computational prediction of rate constants within sufficiently short times suitable for enzyme engineering excludes conventional quantum chemical methods or extensive structural sampling to derive free energy profiles.

### High-Throughput Assay to Acquire Datasets for Developing the Computational Models

For model development or model training or configuration, curated, reproducible datasets are important to ensure reliability. Some embodiments comprise steps relating to the generation and representation of sequence-activity data for model development. FIG. 3 illustrates a high throughput colorimetric assay reaction—e.g. based on 2,2′-azino-bis(3-ethylbenzothiazoline-6-sulfonic acid)) (ABTS)/horseradish peroxidase (HRP)—large quantities of enzyme activity data were generated quickly and efficiently. The data was validated by a secondary enzyme activity assay. That activity may be quantified via high performance liquid chromatography (HPLC). In FIG. 3, The method then involves finding good/neutral positions that are positions found to have mutations which can improve the enzyme activity by at least 0.8 fold against a predetermined substrate—e.g. substrate 152. The method may also involve locating dead positions which are positions that produce mutations that have low activity—e.g. activity of less than 0.8 fold against substrate 152. To use the generated colorimetric data for model development, a method was developed to integrate kinetic (initial rate) and yield (max absorbance) data into enzyme activity values through the following equation: Enzyme Activity=(Initial Rate)×(Max Absorbance)×100. This was validated by the positive linear correlation (R²=0.91) between ln(activity) and ketone yields quantified via HPLC as shown in FIG. 4.

### Sequence Conservation Analysis to Reduce Enzyme Search Space

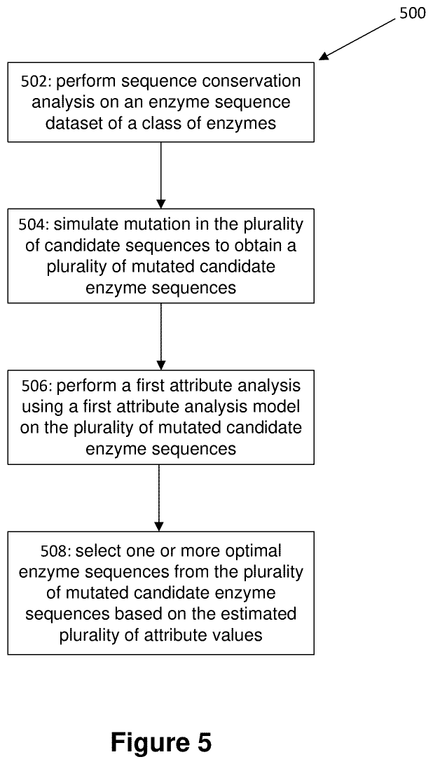

FIG. 5 discloses methods of enzyme evolution (500) which comprise performing sequence conservation analysis on an enzyme sequence dataset of a class of enzymes (step 502). The embodiments have been described by reference to the enzymes of the class of enzymes related to Galactose oxidase (GOase). The methods and systems of the disclosure may be extended to other classes of enzymes to perform enzyme evolution. Sequence conservation analysis comprises selection of a plurality of candidate enzyme sequences in the sequence dataset, wherein the candidate enzyme sequences are viable candidates for subsequent evolution. This may be followed by simulation of mutations in the candidate enzyme sequences to obtain a plurality of mutated candidate enzyme sequences that present a promising starting point for subsequent evolution (step 504). Attribute analysis is performed using one or more attribute analysis models to obtain attributes relating to the mutated candidate enzyme sequences (step 506). One or more optimal enzyme sequences from the plurality of mutated candidate enzyme sequences may be selected based on the estimated attribute values (step 508).

The attribute analysis model may include an aggregation potential prediction model to predict an aggregation potential of mutated candidate enzyme sequences. The attribute analysis model may include an allosteric effect prediction model to predict allosteric effects on fitness of mutated candidate enzyme sequences. The attribute analysis model may include a thermostability prediction model to predict thermostability of mutated candidate enzyme sequences. The attribute analysis model may include a reaction kinetic prediction model to predict kinetics of a reaction of the mutated candidate enzyme sequences. The attribute analysis model may include a catalytic activity prediction model to predict catalytic activity of the mutated candidate enzyme sequences.

The enzyme landscape search could be sped up by identifying positions selected for experimental scanning. For the second batch of saturation mutagenesis in GOase, 200 positions out of 464 positions were selected in Domain I, II and III of GOase. Shannon entropy was used to estimate the diversity in the protein sequence alignment and determine which positions are more or less conserved. Once the entropy threshold was identified, 200 positions were selected which were less conserved and could potentially improve enzyme function once mutated. The selection of 200 positions is merely exemplary. In alternative embodiments, other numbers of positions may be selected. From these 200 prioritized positions, 92 positions were validated experimentally through colorimetric screening. As shown in FIG. 6, among 92 positions, 62 positions were found to produce good/neutral mutants when mutated to all 19 other amino acids while 30 were poor/dead. When compared to predictions, 58 out of 62 positions were predicted positives while 12 out of 30 positions were predicted negatives, giving a prediction accuracy of 76%.

### Aggregation Potential Prediction Model

Analysis of aggregation potential of enzyme variants/mutants was performed with two computational tools/models. In some embodiments the computational model for predicting the aggregation potential of an enzyme variant comprise WALTZ (Oliveberg, M. Waltz, an exciting new move in amyloid prediction. Nat Methods 7, 187-188 (2010). https://doi.org/10.1038/nmeth0310-187), which applies a position specific matrix (PSSM) generated from a large group of hexapeptides. In addition to the PSSM, 19 physical properties of amino acids (such as B-structure propensity and hydrophobicity) were also selected to predict amyloidogenicity.

In some embodiments the computational model for predicting the aggregation potential of an enzyme variant comprised TANGO (Fernandez-Escamilla A M, Rousseau F, Schymkowitz J, Serrano L. Prediction of sequence-dependent and mutational effects on the aggregation of peptides and proteins. Nat Biotechnol. 2004 October; 22 (10): 1302-6. doi: 10.1038/nbt1012. Epub 2004 Sep. 12. PMID: 15361882), which uses a statistical mechanics approach to make secondary structure predictions. Similar physico-chemical parameters are applied and supplemented by the assumption that an amino acid is fully buried in the aggregated state. Designed to predict β-sheet aggregation of proteins, which is different from amyloid fibril formation tendency but usually correlated.

In an experiment, 15 out of 26 mutants validated experimentally were predicted to reduce aggregation by either WALTZ or TANGO, as indicated by the negative score of TANGO and WALTZ when compared to GOh1052. Out of those 15 mutants, 7 were predicted negatively by both TANGO and WALTZ, and two specific mutations, GOh3004 and GOh2056, were found to improve solubility of enzyme by more than 2× of original (exemplary data in Table 1).

### Allosteric Effect Prediction Model

In some embodiments the computational model for predicting allosteric effects on enzyme fitness comprises AlloSigMA, which provides estimation of allosteric free energies acting on a single residue due to either ligand binding, mutations, or both. To understand the effect of a mutation, it is modelled by modifying the strength of the interactions in the mutated residue's contact network. Mutations are categorized into either UP or DOWN mutations, where an UP mutation refers a residue change to a bulkier/heavier amino acid, while a DOWN mutation involves a change to a smaller amino acid. As a result, the dynamic potential of the network changes by making it tighter or looser, depending on the mutation that took place. In an experiment, 17 mutations with distance >15 Å from Cu atom were validated experimentally by comparing the HPLC yields at 24 hrs. Since these mutations were far from the active site, they were shown to possibly have allosteric effect due to the resulting high yield when mutation occur. Out of 17 mutations, AlloSigMA predicted 7 to have a more negative score than GOh1052 backbone which indicated more local stabilization. Two particular mutations, GOh2070 and GOh2044, showed very good activity with 85% HPLC yield and supported by a more negative allosteric score (exemplary data is illustrated in Table 2).

### Thermostability Prediction Model

In some embodiments the computational model for predicting thermostability leverages two tools for modelling stability of enzyme when mutations are introduced-FoldX (https://foldxsuite.crg.eu/) and Rosetta ddg_monomer (https://www.rosettacommons.org/). Some embodiments comprise a hybrid model that automatically integrates Yasara (http://www.yasara.org/) with FoldX for stability prediction of mutants. The hybrid model makes use of Yasara to introduce mutations and perform an energy minimization step before calculating the stability score (ddG) in FoldX.

In some embodiments the Rosetta ddg_monomer application from the Rosetta Commons Suite is incorporated to predict the change in stability (ddG) of a monomeric protein induced by a point mutation. There are two computational steps in the protocol—a preminimization step and the scoring of ddG. For both methods, a negative ddG indicates that the mutant is more thermostable than wildtype while a positive ddG indicates that it is less thermostable. Mutations with distance >15 Å from Cu atom were predicted for thermostability and 33 mutants were selected for experimental validation. From the thermostability assay, 12 mutants showed improvement in residual activity and 9 of which are predicted to be thermostable by either one or both thermostability protocols. In the example of GOase, the top 3 performers predicted and validated experimentally were: GOh2008, GOh2070 and GOh2014 (illustrated in Table 3).

### Reaction Kinetics Prediction Model

In some embodiments the computational method automatically generates structure ensembles with molecular dynamics (MD) simulations of relevant ground states of a relevant catalyzed reaction, i.e. reactants, products and intermediates, based on initial structures of the active site constructed computationally from an experimental X-ray crystal structure (PDB ID: 2EIE). The orientation of the galactose substrate on the ligand was guided from proposed mechanisms reported in literature, aligning the hydroxyl and adjacent CH group with the phenolic groups of Tyr495 and Tyr272, respectively, shown in FIG. 7. The galactose substrate was replaced with ethanol as a model structure for a primary alcohol to reduce model size for demanding DFT calculations. The entire galactose substrate was considered in the model used in subsequent MD simulations. Similar computations may be performed in relation to other enzymes classes considered for evolution.

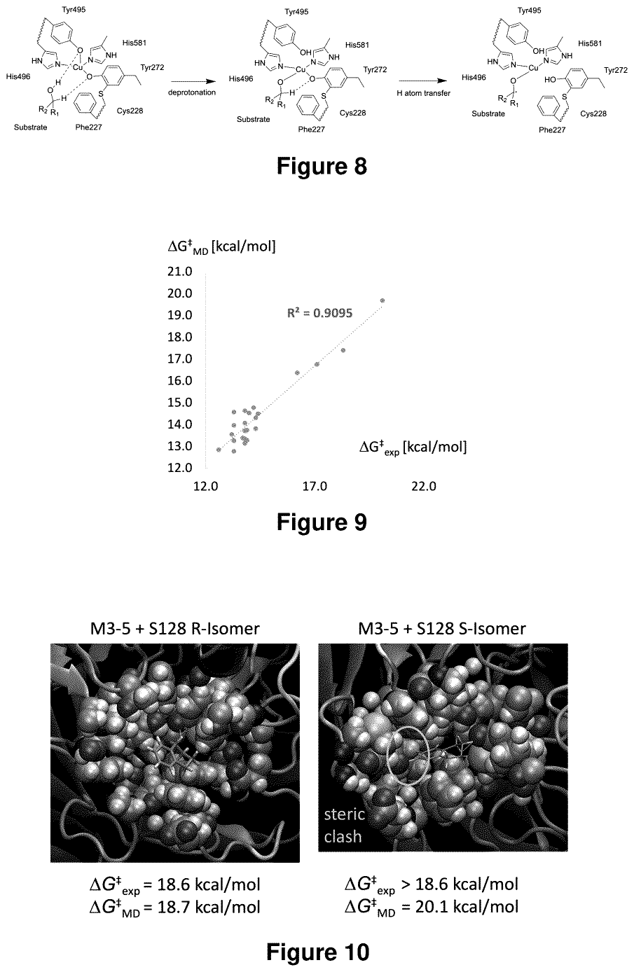

Energies of local minima and transition states of the multi-step oxidation of the model primary alcohol (ethanol) were then investigated using first-principle density functional theory (DFT) calculations using Q-Chem 5.2. The investigated oxidation reaction is summarized in FIG. 8. Geometric structures of reactants, intermediates, and products were verified to be local minima, that is, structural ground states using harmonic frequency analysis, with all calculated vibrational frequencies asserted to be real. Transition state structures that connected local minima were verified to be first-order transition states with one and only one imaginary frequency from harmonic frequency analysis. The rate-determining step was assigned as the step with the highest DFT-calculated Gibbs free-energy difference between transition state and reactant intermediate from the reaction scheme described above. This was determined to be the H atom transfer step. Structures of the local minima (reactant state, RS and product state, PS) and transition state (TS) for the H atom transfer step were then used to construct the enzyme-substrate models in subsequent MD simulations and analyses. Atomistic structures of the RS, PS and TS enzyme-substrate complex were constructed from X-ray crystal structures of the wild type enzyme, with the substrate orientated in their catalytically active state as guided from DFT-calculated active site structures. Structures of the mutant enzymes were then generated using SCWRL4.0. SCWRL is a program for prediction of protein structures, specifically designed for the task of prediction of side-chain conformations given a fixed backbone. New substrates are oriented inside the active site in their catalytically active state by superimposing the relevant functional group moieties of the new substrate (i.e. C—H adjacent to alkoxide) that are directly involved in the reaction with the corresponding moiety of the original enzyme-substrate complex (i.e. Tyr272). Vibrational frequency data from above-mentioned DFT harmonic frequency analysis was also used in the calibration of force fields for MD simulations of RS, TS, and PS. Equilibrated structures of the ground states are used to derive estimated transition state structures for the catalyzed reaction through geometrical interpolation. From these transition state structures, various geometric properties of the active site are derived and correlated with measured rate constants using multidimensional linear regression. These prediction models can then be used to predict rate constants of mutants or new substrates for which rate constants have not been measured before. For various mutants and two different substrates an R²-value of about 0.91 could be achieved when comparing predicted with measured free energy barriers as shown in FIG. 9. Moreover, rate constants for different substrate isomers can be resolved as shown for an example in FIG. 10. Similar computations may be performed in relation to other enzymes classes considered for evolution.

### Catalytic Activity Prediction Model

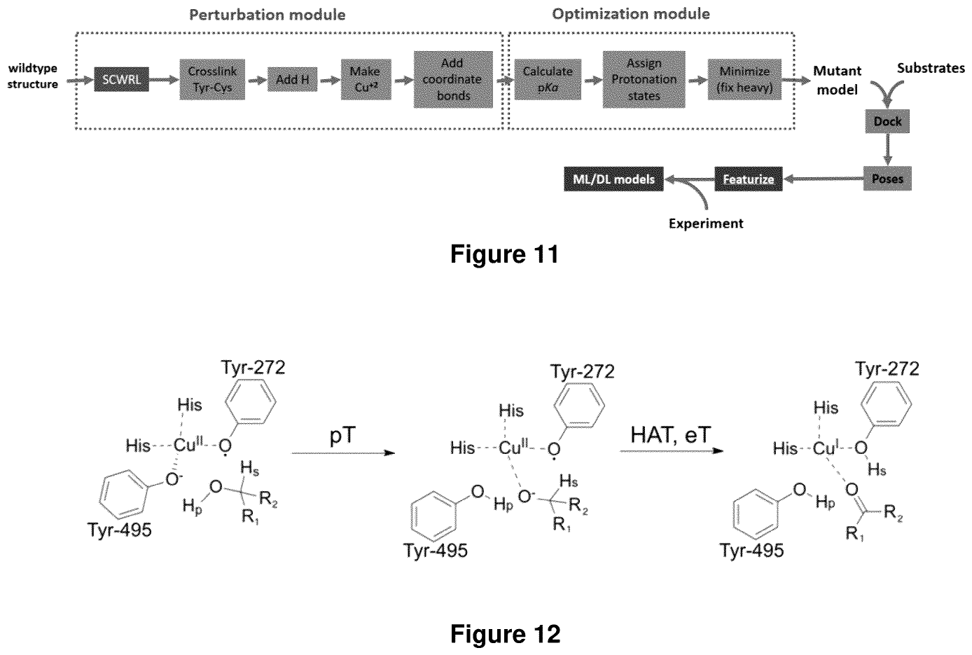

The computational model for predicting catalytic activity of the enzyme variant integrates machine learning and deep learning models with conventional bioinformatics methods for enzyme engineering. The computational model may further include a modelling pipeline an example of which is illustrated in FIG. 11, where GOase mutant models were built with SCWRL, based on available X-ray and quantum-mechanically optimized wild-type GOase structures. Receptors and substrates were modelled and docked in their respective reactive states to capture the correct chemistry and geometry at the catalytic site, using PRIME and GLIDE, respectively: the tyrosyl radical (TYR-O·) was modelled as a fluorinated tyrosine (TYR-F) to closely mimic the vdW radius and atomic charge of the oxygen radical species, while the substrates were modelled in their respective alkoxide (RCHO·) states as illustrated in FIG. 12. These modelled enzyme and substrate states correspond to the rate-determining step GOase catalytic cycle. The docking protocol was optimized to correctly dock to the conformation that ensures hydride transfer in the ensuing step.

The enzyme-substrate complexes from docking calculations were further rescored with one or more scoring functions based on empirical and knowledge-based potentials (MM/GBSA, Glide, Dligand2, Xscore, NNscore2), to better evaluate substrate binding free energies. Docked substrate-receptor pairs were compared against the colorimetric data from experimental training dataset of a selected set of 55 mutants and 64 substrates.

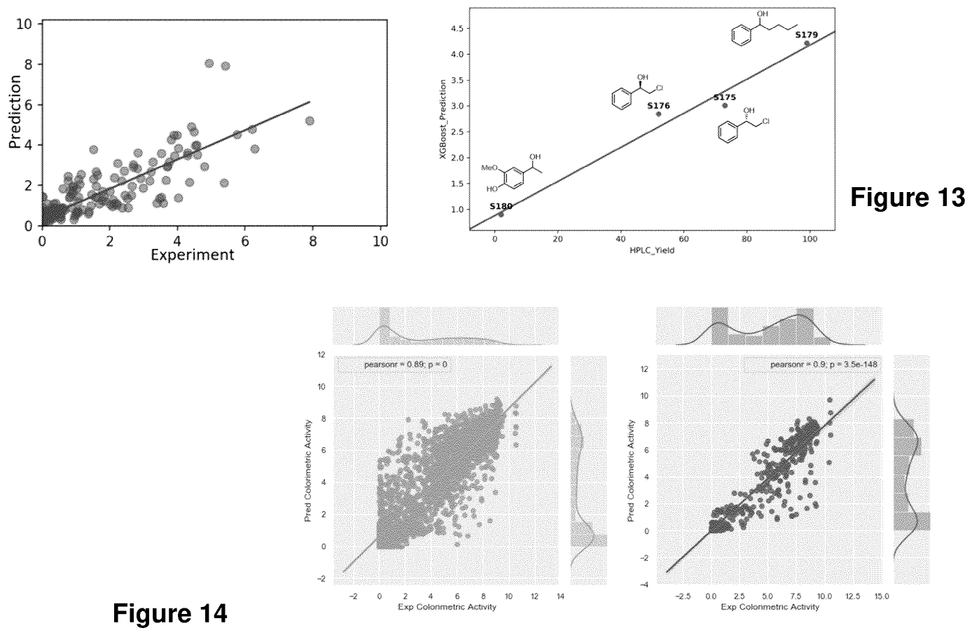

In some embodiments, machine learning models to predict catalytic activity were built using several gradient boosting decision trees approach (XGBoost, CatBoost, LightGBM, and HistGB) to predict catalytic activity using this training set. Numerical features derived from rescoring the docked poses were used, along with several substrate- or enzyme-specific physicochemical features, in all the AI models. To ensure the transferability of the AI models to other enzyme-substrate systems, GOase-specific features were excluded from the feature sets. The model was able to predict increased activity towards substrate of greater chain length using several rescoring schemes and AI models. The machine learning models improved the score correlation with HPLC data with XGBoost outperforming the other models (R^{2~0.89} for test set data) and showed reasonable accuracy (R^{2~0.7}-0.8 for unseen test substrates) for prospective new substrate prediction for variant GOh1052 as illustrated in FIGS. 13 and 14.

Some embodiments incorporate a deep learning model such as a Convolutional Neural Network (CNN) trained to predict the colorimetric activity of mutant-substrate pair. The CNN model inputs are based on the binding energy breakdown from Yasara. In an experiment, a total of 2654 substrate-mutant pairs were used to train the model and the Pearson correlation across the 10-fold cross validation is R=0.89 (R²=0.79). The prediction for a blind test set of 402 substrate-mutant pairs gave a Pearson correlation of R=0.90 (R²=0.81). High correlation with experimental data shows the model's strength in predicting effect of mutants across different substrates as illustrated in FIG. 14.

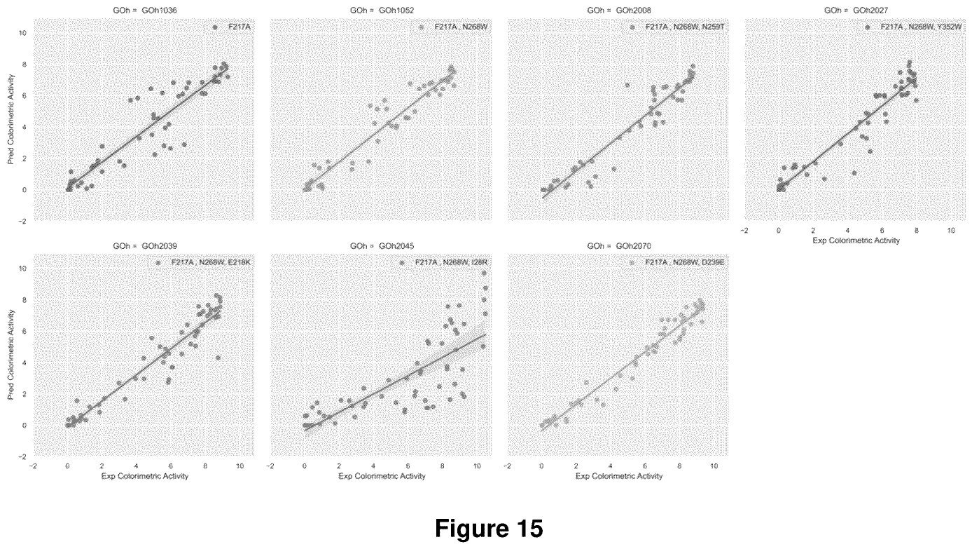

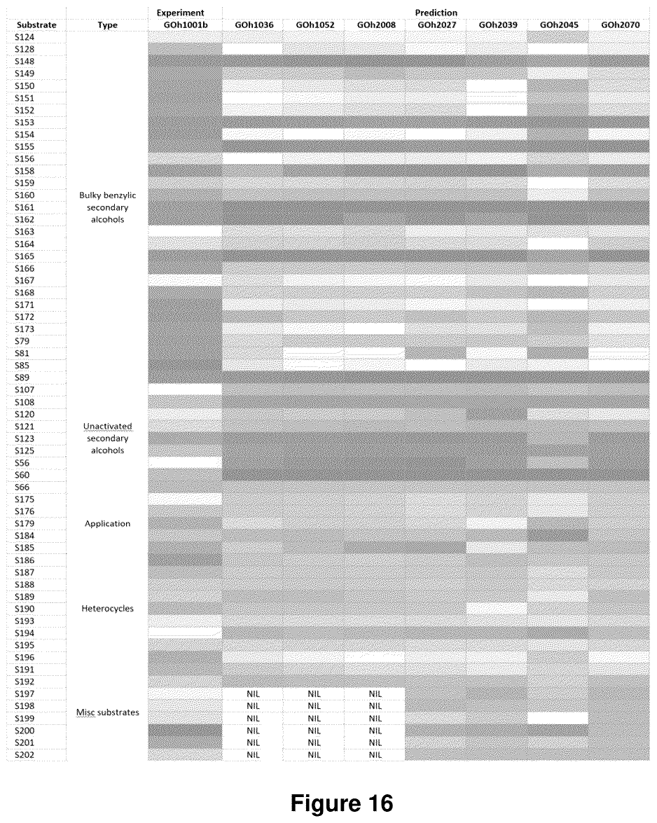

A test set comprised 7 variants (GOh1036, GOh1052, GOh2008, GOh2027, GOh2039, GOh2045 and GOh2070). The Pearson correlation between predicted vs experiment colorimetric activity for each mutant: GOh1036 (r=0.92), GOh1052 (r=0.97), GOh2008 (r=0.96), GOh2027 (r=0.96), GOh2039 (r=0.96), GOh2045 (0.75) and GOh2070 (r=0.98) as illustrated in FIG. 15. GOh1036 was found to be the best single mutant variant when compared to original backbone GOh1001b. GOh1052 was a double mutant found experimentally to be a good generalist for a variety of substrates. GOh2008, GOh2027 and GOh2039 were predicted using our CNN model as good for bulky benzylic substrates with GOh2039 particularly more active on ortho-substituted bulky benzylic secondary alcohols. GOh2045 and GOh2070 showed improvement in activity for heterocycles and application substrates as illustrated in FIG. 16.

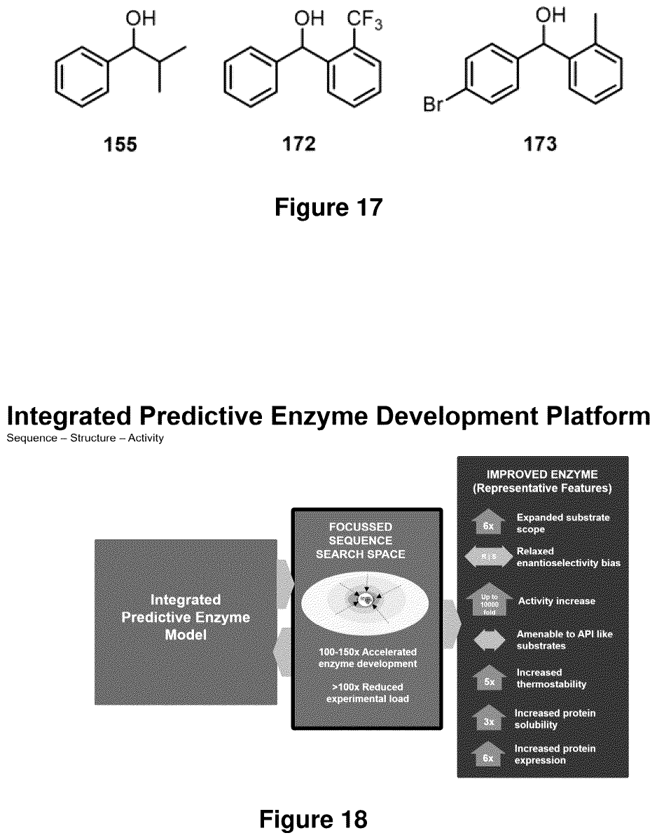

The CNN model was used to predict mutations with improved activity of ortho-substituted substrates. In the experiments, one particular variant (GOh2027) was found to improve binding activity for substrates S155, S172 and S173. GOh2027 gives an improvement of 27% in the HPLC yield for substrate 173 and 12% for substrate S172. The Pearson correlation between the predicted colorimetric activity vs experimental activity is at 0.85 while the correlation between predicted colorimetric activity against HPLC yield is 0.74. The good correlations observed from both activity results shows the extent of model's accuracy in predicting both colorimetric activity as well as for HPLC yields as illustrated in FIG. 17 and Table 4.

In summary, some embodiments of the disclosure provide a high-throughput method to acquire large, curated datasets and a method to correlate measured colorimetric activity (detection of H₂O₂, which is the side product of the biocatalytic oxidation reaction) with the actual enzyme productivity measured by product formation using HPLC (secondary assay). This allows the experimental data to be more effectively used to develop a computational model for attribute analysis in relation to enzyme variants. Some embodiments also incorporate a large panel of high diverse, industrially relevant substrates and scaffolds of which the large, curated datasets are obtained to drive the attribute analysis by the respective computational models. Some embodiments provide a computational model for shortlisting candidate enzyme sequences and recommending good starting points for enzyme engineering/evolution. Some embodiments incorporate two computational models for predicting the aggregation potential of the enzyme variant. Some embodiments include a computational mode for predicting allosteric effects on enzyme fitness. Some embodiments include one or more computational models for predicting the thermostability of the enzyme variant. Some embodiments provide a computational pipeline for predicting activity with the use of ultrashort timeframe simulated annealing molecular dynamic (MD) simulation for energy calculation which is later supplied to a CNN model predictor. The CNN model may be trained with a 2654 or more data points and tested against 402 data points. Some embodiments provide a computational protocol to accelerate the prediction of the rate constants of enzyme mutants with selected substrates in a fully automated simulation workflow based on MD simulations and geometric feature extraction. Some embodiments comprise machine learning models to predict rate constant based on measured rate constants for a subset of enzyme variants and a plurality of substrates. Some embodiments incorporate a computational protocol that integrates protein structural modelling, molecular docking and rescoring, and machine/deep learning models to perform enzyme evolution. The protocol was trained and validated using experimental data measured for 55 mutants and 64 substrates, in total over 3000 data points. The protocol has been fully automated and optimized for high-throughput pipeline. The protocol enabled the prediction of catalytic activity of the enzyme variant for a given substrate.

TABLE 1
Experimental results compared against TANGO/WALTZ predictions.
26 mutants showed improvement in the ratio of the amount
of protein expressed in soluble form against that in
insoluble form (soluble/insoluble) when compared against
the GOh1052 parent. 7 of the mutants were predicted by
both Tango and Waltz to reduce aggregation (green). Top
two performing mutant is GOh3004 and GOh2056 with more
than 2x fold improvement in aggregation.

| | | | | Relative | Soluble/ |
|---|---|---|---|---|---|
| Variant | Mutation | WALTZ | TANGO | Expression | Insoluble |
| GOh3004 | A195S, | −32.78 | −0.72 | 2.42 | 1.15 |
| | N341W | | | | |
| GOh2056 | T265S | −4.01 | −37.22 | 0.65 | 1.12 |
| GOh2079 | Q662D | 0.00 | 0.05 | 1.05 | 1.03 |
| GOh2068 | I485K | 0.00 | 0.10 | 0.54 | 0.79 |
| GOh2080 | S173K | 33.78 | 2.81 | 0.75 | 0.74 |
| GOh2047 | S576D | 0.00 | 0.05 | 0.60 | 0.73 |
| GOh2038 | T429W | 0.00 | 0.00 | 1.34 | 0.72 |
| GOh2067 | I485R | 0.00 | 0.10 | 1.17 | 0.71 |
| GOh2075 | Q662P | 0.00 | 0.00 | 0.55 | 0.71 |
| GOh2073 | S559P | −248.83 | 0.00 | 1.03 | 0.68 |
| GOh2074 | S114N | −540.13 | 0.00 | 0.96 | 0.67 |
| GOh2048 | I284S | −458.19 | 0.00 | 0.52 | 0.66 |
| GOh2046 | I28M | 0.00 | 0.00 | 0.58 | 0.63 |
| GOh2050 | F65P | −731.10 | −6.83 | 0.43 | 0.60 |
| GOh2045 | I28R | −1199.00 | 0.10 | 0.91 | 0.59 |
| GOh2064 | N214R | −231.77 | −1.67 | 0.73 | 0.57 |
| GOh2058 | N214S | −154.52 | 0.00 | 0.95 | 0.53 |
| GOh2061 | I485A | 0.00 | 0.00 | 0.57 | 0.52 |
| GOh2078 | S172E | 33.11 | 0.05 | 1.14 | 0.51 |
| GOh2012 | A195T | −28.09 | −0.72 | 0.95 | 0.50 |
| GOh2055 | A197G | −25.75 | −0.72 | 0.46 | 0.47 |
| GOh2072 | S114R | −495.99 | 0.10 | 1.11 | 0.47 |
| GOh2077 | D239P | 0.00 | −10.01 | 0.62 | 0.46 |
| GOh2001 | A195S | −32.78 | −0.72 | 0.68 | 0.45 |
| GOh2026 | K389I | 0.00 | −0.43 | 1.14 | 0.45 |
| GOh2057 | S220T | 0.00 | 0.02 | 0.15 | 0.45 |
| GOh2066 | D269N | 403.01 | 10.50 | 1.32 | 0.45 |
| GOh3002 | N341W, | −288.96 | 0.00 | 0.89 | 0.45 |
| | Y352T | | | | |
| GOh1052 | — | 0.00 | 0.00 | 1.00 | 0.44 |

TABLE 2
HPLC Yield compared against AlloSigMA scores for mutations
that showed higher activity but are far from the active
site. Two top performing mutants GOh2070 and GOh2044
showed highest improvement in HPLC and predicted to
be more stable in allosteric potential.

| | | Room Temp | | Prediction |
|---|---|---|---|---|
| | | HPLC Yield | Prediction | Allostery |
| Variant | Mutation | (24 h) | Allostery | Type |
| GOh2070 | D239E | 85% | −2.11 | UP |
| GOh2044 | A24H | 85% | −2.15 | UP |
| GOh2014 | N341W | 77% | −1.64 | UP |
| GOh2045 | I28R | 77% | −1.91 | UP |
| GOh2017 | A198I | 70% | −1.91 | UP |
| GOh2076 | S173R | 67% | −1.40 | UP |
| GOh2080 | S173K | 67% | −1.40 | UP |
| GOh2025 | A346L | 67% | −1.69 | UP |
| GOh2019 | T202D | 66% | −1.65 | UP |
| GOh2072 | S114R | 62% | −1.43 | UP |
| GOh2046 | I28M | 61% | −1.91 | UP |
| GOh2051 | F65R | 59% | −1.81 | UP |
| GOh2078 | S172E | 58% | −1.44 | UP |
| GOh2021 | T202I | 58% | −1.65 | UP |
| GOh2074 | S114N | 57% | −1.43 | UP |
| GOh2053 | T153R | 57% | −1.68 | UP |
| GOh2047 | S576D | 56% | −1.61 | UP |
| GOh1052 | — | 55% | −1.68 | — |

TABLE 3
Experimental results of 12 mutants showing improvement in residual activity
as compared to backbone GOh1052 (red). Out of 9 mutants predicted correctly
to be thermostable, 3 mutants (green) produced negative score for both methods
(YASARA + FoldX and Rosetta ddg_monomer).

| | | | 50 deg | 50 deg | | |
|---|---|---|---|---|---|---|
| GOh | | | Heated HPLC | Residual | YASARA + | Rosetta |
| Series | Mutation | Distance | Yield (24 h) | Activity | FoldX | ddg_monomer |
| GOh2008 | N259T | >15 | 54% | 84% | −0.71 | −2.96 |
| GOh2045 | I28R | >15 | 61% | 79% | 0.00 | 6.25 |
| GOh2017 | A198I | >15 | 55% | 79% | −2.81 | 0.19 |
| GOh2070 | D239E | >15 | 66% | 78% | −0.53 | −4.48 |
| GOh2021 | T202I | >15 | 43% | 74% | 0.18 | −1.48 |
| GOh2044 | A24H | >15 | 63% | 74% | 0.44 | −0.25 |
| GOh2046 | I28M | >15 | 43% | 70% | 0.54 | 3.01 |
| GOh2014 | N341W | >15 | 52% | 68% | −0.93 | −2.67 |
| GOh2043 | I284Y | >15 | 41% | 65% | 0.51 | −0.49 |
| GOh2048 | I284S | >15 | 38% | 62% | 1.36 | 2.89 |
| GOh2050 | F65P | >15 | 36% | 61% | −7.77 | 3.84 |
| GOh2053 | T153R | >15 | 33% | 58% | −0.94 | 3.00 |
| GOh1052 | — | — | 31% | 56% | 0.00 | 0.00 |

TABLE 4
Experiment and prediction results for variant GOh2027
vs backbone GOh1052 across the three ortho-substituted
substrates - S155, S172 and S173.

| | | | | Exp | Pred |
|---|---|---|---|---|---|
| | | | | Color- | Color- |
| | | | HPLC | imetric | imetric |
| Substrate | Variant | Mutation | (%) | Activity | Activity |
| S155 | GOh1052 | F217A, N268W | 4.0 | 1.02 | 0.17 |
| S172 | GOh1052 | F217A, N268W | 6.0 | 2.77 | 1.70 |
| S173 | GOh1052 | F217A, N268W | 60.0 | 4.86 | 4.26 |
| S155 | GOh2027 | F217A, N268W, | 5.0 | 0.10 | 0.30 |
| | | Y352W | | | |
| S172 | GOh2027 | F217A, N268W, | 18.0 | 0.96 | 1.45 |
| | | Y352W | | | |
| S173 | GOh2027 | F217A, N268W, | 87.0 | 5.30 | 2.43 |
| | | Y352W | | | |

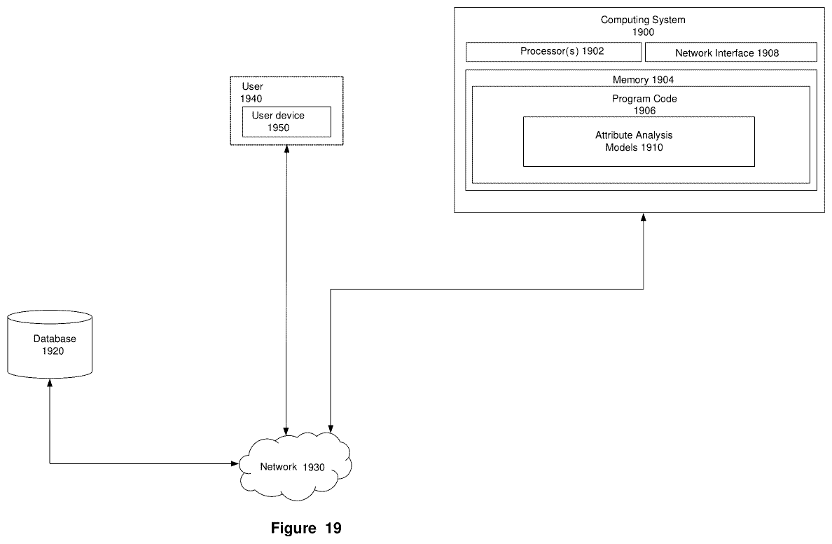

FIG. 19 illustrates a block diagram of a system for enzyme evolution 1900. According to FIG. 19, the system comprises at least one processor 1902, memory 1904 accessible to the processor 1902 and a network interface 1908 to facilitate communication with a database 1920 and a user computer device 1950 of a user 1940. Program code 1906 provided in memory 1904 comprises instructions executable by the processor 1902 to perform at least a part of the method of the embodiments described herein.

Notably, while individual computer systems are described in FIG. 19, any such computer system may be distributed across multiple servers or multiple devices, or some functionality may be consolidated into a single server or device, without departing from the purposive intent of the present disclosure.

The users device 1950 can facilitate extraction of data (e.g. an enzyme sequence dataset of a class of enzymes) from databases 1920, and implementation of framework 100, or initiation of method 500, by processor(s) 1902 of computing system 1900. The users device 1950 may comprise a software application for prediction of protein structures and generating enzyme mutants, for example, protein structures of the mutant enzymes stored in the database 1920. The user computing device 1950 may be a personal computer. Network 1930 facilitates communication between the various devices (e.g. user device 1950 and database 1920) and may include one or more communication networks including the internet, ethernet, etc.

The system 1900 first perform sequence conservation analysis on an enzyme sequence dataset of a class of enzymes to select a plurality of candidate sequences in the sequence dataset stored in database 1920. It then simulate mutation in the plurality of candidate sequences to obtain a plurality of mutated candidate enzyme sequences using computer software stored in user device 1950. The system then performs attribute analysis using a plurality of attribute analysis models 1910 on the plurality of mutated candidate enzyme sequences to estimate a plurality of attribute values of each of the plurality of mutated candidate enzyme sequence. The system then selects one or more optimal enzyme sequences from the plurality of mutated candidate enzyme sequences based on the estimated plurality of attribute values.

One or more database 1920 are also accessible to the system 1900. Each database 1920 may comprises records such as one or more enzyme sequence dataset of a class of enzymes used in method 500.

The plurality of attribute analysis models 1910 may comprise of any one of aggregation potential prediction model to predict an aggregation potential of mutated candidate enzyme sequences, allosteric effect prediction model to predict allosteric effects on fitness of mutated candidate enzyme sequences, thermostability prediction model to predict thermostability of mutated candidate enzyme sequences, reaction kinetic prediction model to predict kinetics of a reaction of the mutated candidate enzyme sequences, catalytic activity prediction model to predict catalytic activity of the mutated candidate enzyme sequences, or combinations thereof.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Although this disclosure describes or illustrates particular embodiments as providing particular advantages, particular embodiments may provide none, some, or all of these advantages.

1. A system of enzyme evolution, the system comprising:
a memory accessible to processor(s), the memory comprising program code executable by the processors to:
perform sequence conservation analysis on an enzyme sequence dataset of a class of enzymes to select a plurality of candidate sequences in the sequence dataset;
simulate mutation in the plurality of candidate sequences to obtain a plurality of mutated candidate enzyme sequences;
perform a first attribute analysis using a first attribute analysis model on the plurality of mutated candidate enzyme sequences to estimate a first attribute value of each of the plurality of mutated candidate enzyme sequence;
select one or more optimal enzyme sequences from the plurality of mutated candidate enzyme sequences based on the estimated first attribute values.

2. A system for enzyme evolution, the system comprising:
a memory accessible to processor(s), the memory comprising program code executable by the processors to:
perform sequence conservation analysis on an enzyme sequence dataset of a class of enzymes to select a plurality of candidate sequences in the sequence dataset;
simulate mutation in the plurality of candidate sequences to obtain a plurality of mutated candidate enzyme sequences;
perform attribute analysis using a plurality of attribute analysis models on the plurality of mutated candidate enzyme sequences to estimate a plurality of attribute values of each of the plurality of mutated candidate enzyme sequence;
select one or more optimal enzyme sequences from the plurality of mutated candidate enzyme sequences based on the estimated plurality of attribute values;
wherein each of the plurality of attribute analysis models comprises any one of:
aggregation potential prediction model to predict an aggregation potential of mutated candidate enzyme sequences; or
allosteric effect prediction model to predict allosteric effects on fitness of mutated candidate enzyme sequences;
thermostability prediction model to predict thermostability of mutated candidate enzyme sequences; or
reaction kinetic prediction model to predict kinetics of a reaction of the mutated candidate enzyme sequences; or
catalytic activity prediction model to predict catalytic activity of the mutated candidate enzyme sequences.

3. A method for enzyme evolution, the method comprising:
performing sequence conservation analysis on an enzyme sequence dataset of a class of enzymes to select a plurality of candidate enzyme sequences in the sequence dataset, wherein the candidate enzyme sequences are viable candidates for subsequent evolution;
simulating mutation in the plurality of candidate enzyme sequences to obtain a plurality of mutated candidate enzyme sequences;
performing a first attribute analysis using a first attribute analysis model on the plurality of mutated candidate enzyme sequences to estimate a first attribute value of each of the plurality of mutated candidate enzyme sequence;
selecting one or more optimal enzyme sequences from the plurality of mutated candidate enzyme sequences based on the estimated first attribute values.

4. The method as claimed in claim 3, wherein the method further comprises:
performing a second attribute analysis using a second attribute analysis model on the plurality of mutated candidate enzyme sequences to estimate a second attribute value of each of the plurality of mutated candidate enzyme sequence;
wherein the one or more optimal enzyme sequences are selected based on the estimated first attribute values and the estimated second attribute values.

5. The method as claimed in claim 4, wherein the first and second attribute analysis models comprise any one of:
aggregation potential prediction model to predict an aggregation potential of mutated candidate enzyme sequences; or
allosteric effect prediction model to predict allosteric effects on fitness of mutated candidate enzyme sequences;
thermostability prediction model to predict thermostability of mutated candidate enzyme sequences; or
reaction kinetic prediction model to predict kinetics of a reaction of the mutated candidate enzyme sequences; or
catalytic activity prediction model to predict catalytic activity of the mutated candidate enzyme sequences.

6. The method as claimed inclaim 3, wherein the sequence conservation analysis is performed by computing entropy metrics in relation to each of the mutated candidate enzyme sequences to estimate diversity in protein sequence alignment and estimate sequence conservation based on the estimate diversity.

7. The method as claimed in claim 3, wherein the first attribute indicates aggregation potential of the respective candidate sequences; and
the first attribute analysis model is configured to predict at least one of: amyloidogenicity or β-sheet aggregation of the mutated candidate enzyme sequences to determine the aggregation potential.

8. The method as claimed in claim 3, wherein the first attribute indicates allosteric effect of the respective candidate sequences; and
the first attribute analysis model is configured to predict the allosteric free energy of the mutated candidate enzyme sequences to determine the first attribute.

9. The method as claimed in claim 3, wherein the first attribute indicates allosteric effect of the mutated candidate enzyme sequences; and
the first attribute analysis model is configured to predict the allosteric free energy of the mutated candidate enzyme sequences to determine the first attribute.

10. The method as claimed in claim 3, wherein the first attribute indicates thermostability of the mutated candidate enzyme sequences; and
the first attribute analysis model is configured to perform energy minimization computation with respect to the mutated candidate enzyme sequences and calculate stability of the mutated candidate enzyme sequences to determine the first attribute.

11. The method as claimed in claim 3, wherein the first attribute indicates kinetics of the mutated candidate enzyme sequences; and
the first attribute analysis model is configured to predict rate constants associated with the mutated candidate enzyme sequences in relation to a specific reaction to determine the first attribute.

12. The method as claimed in claim 11, wherein the first attribute analysis model comprises a multidimensional regression model obtained by simulation of transition state structures of a wild type enzyme of the class of enzymes in relation to the reaction to obtain geometric properties active sites and a correlation of the geometric properties with reaction kinetics.

13. The method as claimed in claim 3, wherein the first attribute indicates catalytic activity of the mutated candidate enzyme sequences; and
the first attribute analysis model is configured to:
simulate docking of a substrate in relation to the mutated candidate enzyme sequences to obtain geometric properties of the mutated candidate enzyme sequences; and
estimate the first attribute based on a machine learning model trained using calorimetric activity of a subset of the plurality of the candidate enzyme sequences and substrate combinations.

14. The method as claimed in claim 13, wherein the machine learning model comprises a deep learning model.

15. The method as claimed in claim 3, wherein the method further comprises generating enzyme sequence-activity data in relation to a subset of the plurality of candidate sequences using a colorimetric assay;
wherein the first attribute analysis model performing a first attribute analysis based on the generated enzyme sequence-activity data.

16. (canceled)
